(19) United States
(12) Patent Application Publication
(10) Pub. No.: US 2022/0278674 A1
(43) Pub. Date: Sep. 1, 2022

(54) HIGH VOLTAGE PULSE GENERATOR FOR ELECTRO-PORATION

(71) Applicant: IGEA S.P.A., CARPI (MO) (IT)

(72) Inventors: Ruggero CADOSSI, CARPI (MO) (IT); Donata MARAZZI, CARPI (MO) (IT); Claudio BERTACCHINI, CARPI (MO) (IT)

(21) Appl. No.: 17/680,742

(22) Filed: Feb. 25, 2022

(30) Foreign Application Priority Data
Feb. 26, 2021 (IT) 102021000004601

(51) Int. Cl. H03K 3/282; A61N 1/02; A61N 1/04

(52) U.S. Cl. CPC H03K 3/2821; A61N 1/025; A61N 1/0412

(57) ABSTRACT

A high voltage pulse generator for electro-poration comprising: a capacitor bank in which the electric charge used to generate the pulses is stored; the capacitor bank has an output terminal on which a high voltage direct current HT is present; a voltage amplifier circuit which receives, at input, the high voltage direct current HT and is provided with an automatic gain control circuit designed to compare a reference voltage V_{HT-ref} with the voltage supplied at output V_{OUT} by the amplifier to maintain the voltage constant at output during generation of the pulses; a current amplifier circuit arranged downstream of the voltage amplifier circuit and provided with an electronic semiconductor switch communicating with a first terminal with the output of the capacitor bank and provided with a second terminal designed to communicate with an output of the pulse generator; the voltage present at the output of the voltage amplifier is supplied to the output of the pulse generator and is used to operate the switching during closing of the electronic semiconductor switch.

### CROSS-REFERENCE TO RELATED APPLICATIONS

This patent application claims priority from Italian patent application no. 102021000004601 filed on Feb. 26, 2021, the entire disclosure of which is incorporated herein by reference.

### TECHNICAL FIELD

The present invention relates to a high voltage pulse generator for electro-poration.

### BACKGROUND ART

As it is known, electro-poration treatments involve the application of high voltage electrical pulses (about 1000 Volts) produced by a pulse generator to an organic tissue by means of the use of electrodes applied to the tissue itself; the electric field generated in the tissue produces the formation of pores in the cell plasma membrane causing a change in its permeability which facilitates the flow of organic/inorganic substances (such as DNA or medicinal products) from the outside to the inside of the cell. These electro-poration treatments are controllable based on the parameters (voltage, waveform, duty-cycle, application time, number of pulses applied, etc.) of the electrical pulses. In some applications the electrodes are inserted into the tissue to make the electro-poration process more effective; for example, the electrodes can comprise a plurality of needles that are electrically connected with the pulse generator and that are inserted inside the portion of the body of a patient to which the electro-poration treatment must be applied.

Pulse generators for electro-poration of the known type comprise a capacitor bank in which the electric charge used to generate the pulses is stored and an electronic switch which is switched by a driving signal to produce a given waveform of the pulses, for example a square wave.

The pulse generators of the known type, however, produce a waveform that is strongly dependent on the impedance of the organic tissue and for this reason the amplitude of the pulses in many operating conditions do not correspond to that expected, especially if the current absorbed by the tissue noticeably varies during the electro-poration treatment following the variation of the impedance, in particular following the decrease of the impedance. Document WO 2017/117251 discloses an electro-poration device.

### DISCLOSURE OF INVENTION

The object of the present invention is to manufacture a pulse generator that allows good control of the pulse voltage even if the current absorbed by the load varies significantly following the variation of the impedance during treatment.

The preceding object is achieved by the present invention since it relates to a high voltage pulse generator for electro-poration comprising: a capacitor bank in which the electric charge used to generate the pulses is stored; the capacitor bank has an output terminal on which a high voltage direct current HT is present; a voltage amplifier circuit which receives, at input, the high voltage direct current HT and is provided with an automatic gain control circuit able to compare a reference voltage V_{HT-ref} with the voltage supplied at output V_{OUT} by the amplifier to keep the output voltage constant during the generation of the pulses; a current amplifier circuit arranged downstream of the voltage amplifier circuit and provided with an electronic semiconductor switch communicating with a first terminal with the output of the capacitor bank and provided with a second terminal designed to communicate with an output of the pulse generator; the voltage present at the output of the voltage amplifier is supplied to the pulse generator output and is used to switch-off said electronic semiconductor switch.

### BRIEF DESCRIPTION OF THE DRAWINGS

The invention will now be illustrated with reference to the attached drawings which represent a preferred non-limiting embodiment thereof wherein:

FIG. 1 illustrates an electrical diagram of a high voltage pulse generator for electro-poration made according to the present invention; and

FIG. 2 illustrates pulses obtained from a known type of pulse generator; and

FIG. 3 illustrates experimental data illustrating the waveform produced by the pulse generator according to the present invention.

### BEST MODE FOR CARRYING OUT THE INVENTION

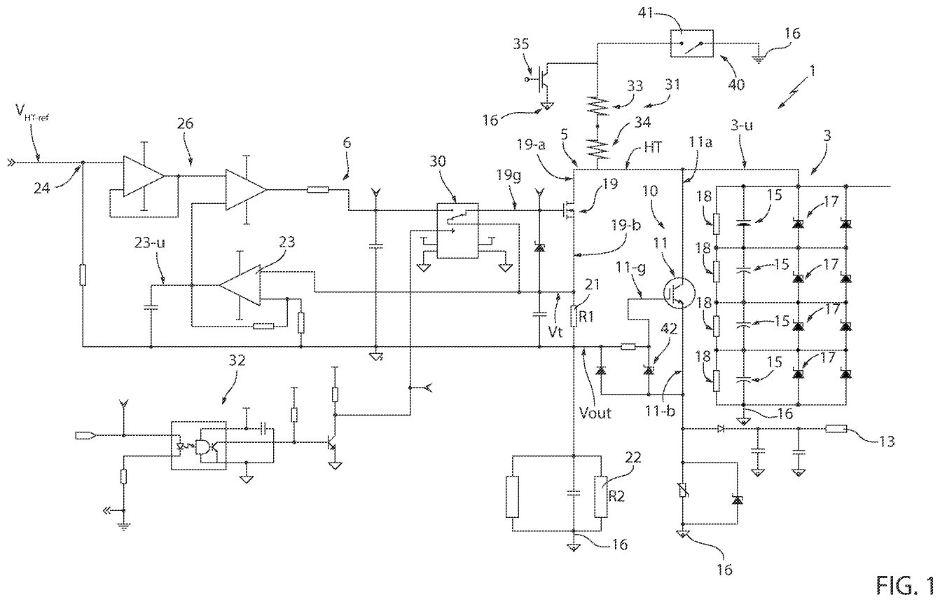

In FIG. 1, 1 denotes, as a whole, a high voltage pulse generator for electro-poration. The terms high voltage pulses refer to pulses having a voltage of the order of about 1000 Volts (in some applications the pulses can reach values of 3000 V).

The pulse generator 1 comprises:

a capacitor bank 3 in which the electric charge used to generate the pulses is stored; the capacitor bank 3 has an output terminal 3u on which a high voltage direct current HT is present (for example 1200 Volt).

A voltage amplifier circuit 5 which receives, at input, the high voltage direct current HT and is provided with an automatic gain control circuit 6 designed to compare a reference voltage V_{HT-ref} with an output voltage Vr from the amplifier 5 to keep the output voltage V_{OUT} constant during the generation of the pulses;

a current amplifier circuit 10 arranged downstream of the voltage amplifier circuit 5 and provided with an electronic semiconductor switch 11 communicating with its first terminal 11-a with the output terminal 3u of the capacitor bank 3 and provided with a second terminal 11-b designed to communicate with an output 13 of the pulse generator 1;

the voltage V_{OUT} present at the output of the voltage amplifier 5 is supplied to the output of the pulse generator (less than a small potential drop ΔZ of a few volts) and is used to switch-off the electronic semiconductor switch 11.

An electrode for electro-poration (of a known type and not shown) can be connected to the output 13 and applied to a patient which represents a load for the pulse generator 1. a) The voltage applied to the tissue in clinical practice is always expressed as V/cm.

Preferably the capacitor bank 3 is formed by a plurality of electrolytic capacitors 15 arranged in series with one another between a reference terminal 16 (ground) and the output terminal 3u. For example, four 1000 μF 400Volt capacitors can be used. Each capacitor 15 is provided with a respective protection and charge balancing circuit formed respectively by a Zener diode 17 (or by a pair of Zener diodes arranged in parallel as illustrated) and by a resistor 18 arranged in parallel with the capacitor 15. A typical resistance of resistor 18 value is 10 MOhm. A non-limiting example of the Zener diode 17 is the p6ke440a. The charging voltage of the capacitor bank 3 can be detected by means of a resistive divider (not illustrated) and an AD converter designed to measure the voltage present on the resistive divider. In turn, the capacitors 15 are charged by a voltage boosting circuit of a known type (not illustrated).

The voltage amplifier circuit 5 comprises a second electronic switch 19 (in the example a power MOSFET) having a first terminal 19-a communicating with the output terminal 3u of the capacitor bank 3 and a second terminal 19-b connected with the reference terminal 16 (ground) via a first resistor 21 (resistance value R1) and a second resistor 22 (resistance value R2 much higher than R1 for example R1=10 Ohm R2=100 KOhm) making a voltage divider; the automatic gain control circuit 6 is configured to compare the voltage Vr present on the first resistor 21 with the reference voltage V_{HT-ref} to produce a driving signal of the second electronic switch 19, in the example the signal supplied to the gate 19g of the MOSFET. The voltage Vr is in fact proportional to the voltage Vout according to:

Vr=Vout−R2*HT/(R1+R2)

The automatic gain control circuit 6 comprises a non-inverting amplifier 23 which receives, at input, the voltage Vr at the ends of the first resistor 21 and that has an output 23-u in which a voltage is present, which is compared with the reference voltage V_{HT-ref} in an adder node 24.

The reference voltage V_{HT-ref} has a periodic trend (for example square wave) which regulates the waveform and the frequency of the pulse generator signal 1.

The difference between V_{HT-ref} and the output voltage of the non-inverting amplifier 23 is supplied to the input of a follower amplifier 26. The output of the follower amplifier 26 produces the driving signal of the second electronic switch 19.

A circuit breaker switch 30 is arranged between the output of the follower amplifier 26 and the control terminal (gate 19g) of the second electronic semiconductor switch. A control circuit 32 is configured to achieve the opening of the circuit breaker switch 30 when, for any reason, one wishes to turn off the switch 19 and consequently also the switch 10, thereafter interrupting the supply of the driving signal to the second electronic switch 19 that opens instantly.

The pulse generator 1 comprises a controlled discharge circuit 31 of the capacitors 15, so as to reduce the voltage on the capacitors to a desired value without necessarily having to completely discharge the capacitors 15 and then recharge the same, and which connects the output terminal 3u with the ground 16 through of a pair of dissipator resistors 33, 34 connected in series. Said circuit can also be used so as to prevent the risks associated with the high voltage present in the capacitors 15. The activation of the controlled discharge circuit 31 is achieved by sending a closing signal to a third electronic semiconductor switch 35 (for example an IGBT) arranged between the resistor 33 and the ground 16. The third electronic semiconductor switch 35 is open during the operation of the pulse generator 1.

The pulse generator 1 comprises a quick discharge circuit 40 of the capacitors 15 which connects the output terminal 3u with the ground 16 through the resistors 33 and 34 through a quick-closing relay 41. The use of a relay 41 achieves a quick and deep discharge in case of emergency. In fact, it has been seen that the discharge IGBT 35 is unable to discharge resistors 33 and 34 to zero but leaves a residual voltage of a couple of tens of volts.

The electronic semiconductor switch 11 cooperates with a Zener diode 42 interposed between the control terminal 11g (in the example the gate of the IGBT 11) and the second terminal of the electronic switch (emitter of the IGBT) to obtain the appropriate gate voltage. The voltage Vout is applied both to the control terminal 11g and to the cathode of the Zener diode 42. In this way, in the presence of a Vout, the voltage applied between the gate and the emitter of the IGBT 11 corresponds to the voltage of the Zener (for example 9 volts) and is higher than the threshold value of the IGBT which can work in saturation by letting flow all the current required by the load and coming from the capacitors 15. The voltage across the Zener 42 produces the potential drop AZ.

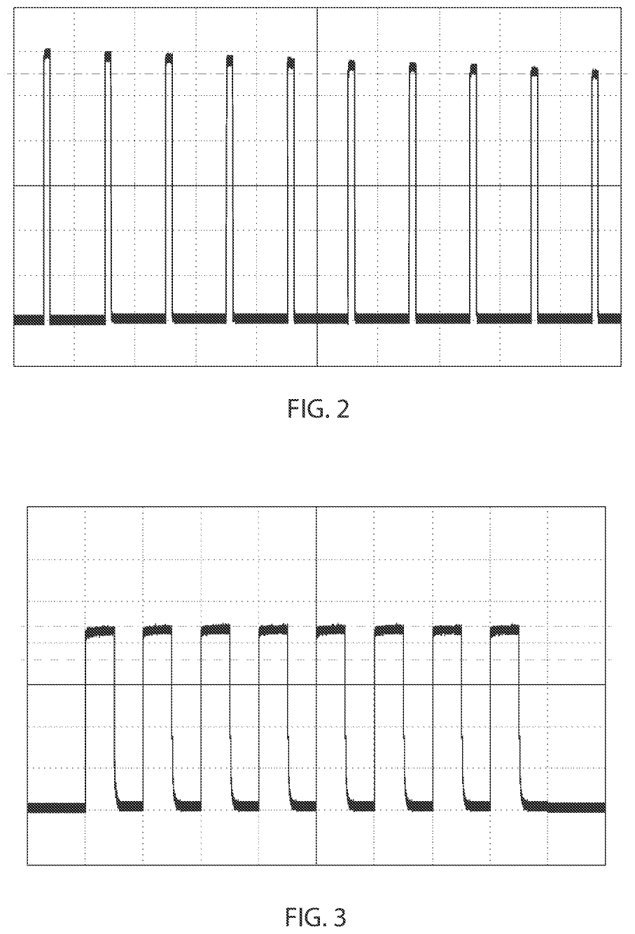

The advantages of the pulse generator 1 are as follows:
obtain pulses with stable amplitude during generation of the same.
Being able to obtain pulses with an arbitrary shape;
the mode of generation of the pulses allows to keep the single pulses largely homogeneous; consider FIG. 2 which illustrates pulses obtained with known technologies and FIG. 3 which illustrates pulses obtained by the pulse generator according to the present invention;
emphasize that the repeatability and homogeneity of the pulses is important to ensure effective electro-poration of all cells in the tissue.

1. A high voltage pulse generator for electro-poration comprising
a capacitor bank (3) in which the electric charge used to generate the pulses is stored; the capacitor bank (3) has an output terminal (3u) on which a high voltage direct current HT is present;
characterized by comprising a voltage amplifier circuit (5) which receives, at input, the high voltage direct current HT and is provided with an automatic gain control circuit (6) designed to compare a reference voltage VHT-ref with the voltage supplied at output V_{OUT} by the amplifier (5) to maintain the voltage constant at output during generation of the pulses;
a current amplifier circuit (10) arranged downstream of the voltage amplifier circuit (5) and provided with an electronic semiconductor switch (11) communicating with a first terminal (11-a) with the output of the capacitor bank (3) and provided with a second terminal (11a) designed to communicate with an output (13) of the pulse generator;
the voltage present at the output of the voltage amplifier (5) is supplied to the output (13) of the pulse generator and is used to operate the switching during closing of said electronic semiconductor switch (11).

2. The pulse generator according to claim 1 wherein the capacitor bank (3) is formed by a plurality of electrolytic capacitors (15) arranged in series with one another between a reference terminal (16, ground) and the output terminal (3u) of the capacitor bank (3); each capacitor (15) is provided with a respective protection and charge balancing circuit formed respectively by a Zener diode (17) and by a resistor (18) arranged in parallel to the capacitor (15).

3. The generator according to claim 1, wherein the voltage amplifier circuit (5) comprises a second electronic semiconductor switch (19) having a first terminal (19-a) communicating with the output terminal (3u) of the capacitor bank (3) and a second terminal (19-b) connected to a reference terminal (16, ground) via a first resistor (21, R1) and a second resistor (22, R2) forming a voltage divider; the automatic gain control circuit (6) is configured to compare the voltage Vr present on the first resistor (21) with the reference voltage V_{HT-ref} to produce a driving signal of the second electronic switch (19).

4. The pulse generator according to claim 3, wherein the automatic gain control circuit (6) comprises a non-inverting amplifier (23) which receives at input the voltage Vr at the ends of the first resistor (21) and which has an output (23-u) which is compared with the reference voltage V_{HT-ref}; the difference between the reference voltage V_{HT-ref} and the output voltage of the non-inverting amplifier (23) is supplied to the input of a follower amplifier (26); the output of the follower amplifier (26) produces the driving signal of the second electronic switch (19).

5. The pulse generator according to claim 4, wherein a circuit breaker switch (30) is arranged between the output of the comparator amplifier (26) and the control terminal (gate 19g) of the second electronic semiconductor switch (19); a control circuit (32) is configured to achieve the opening of the circuit breaker switch (30) when a potentially dangerous situation is detected, thereafter interrupting the supply of the driving signal to the second electronic switch (19) which opens instantaneously.

6. The pulse generator according to claim 2, wherein a controlled discharge circuit (31) of the capacitors (15) is provided, which is configured to arrange in connection the output terminal (3u) of the capacitor bank with the reference terminal (ground 16) through a dissipator resistor (33, 34) and a semiconductor switch (35) after the switching-off of the pulse generator (1).

7. The pulse generator according to claim 1, wherein a quick discharge circuit (40) of the capacitors (15) is provided, which is configured to arrange in connection the output terminal (3u) of the capacitor bank with the reference terminal (ground 16) through a dissipator resistor (33, 34) and a quick-closing relay (41) after the switching-off of the pulse generator (1).

8. The pulse generator according to claim 1, wherein the electronic semiconductor switch (11) cooperates with a Zener diode (42) interposed between a control terminal (11g) and a reference terminal of the electronic switch (11);
the voltage supplied at output V_{OUT} is furthermore applied to the cathode of the Zener diode (42) which has a voltage higher than the threshold voltage of the electronic switch (11) which can work in saturation leaving the current coming from the capacitor bank (15) towards a load of the pulse generator.
